(19) United States
(12) Patent Application Publication
(10) Pub. No.: US 2026/0083469 A1
(43) Pub. Date: Mar. 26, 2026

(54) OPERATOR-INDEPENDENT HISTOTRIPSY DEVICE

(71) Applicant: QUANTANOSIS.AI, INC., Harlingen, TX (US)

(72) Inventors: Ameer E. Hassan, Harlingen, TX (US); Yousef Hasan Ahmad Khalili, Dubai (AE)

(21) Appl. No.: 19/408,930

(22) Filed: Dec. 4, 2025

Related U.S. Application Data
(63) Continuation-in-part of application No. 17/923,274.
(60) Provisional application No. 63/064,008, filed on Aug. 11, 2020.

(51) Int. Cl. A61B 17/22; A61B 8/00; A61B 8/08; A61B 17/00; A61B 90/00

(52) U.S. Cl. CPC A61B 17/22012; A61B 8/085; A61B 8/4218; A61B 8/4444; A61B 8/488; A61B 90/06; A61B 2017/00106; A61B 2017/00154; A61B 2017/00367; A61B 2017/22008; A61B 2017/22014; A61B 2017/22028; A61B 2090/064

(57) ABSTRACT

A histotripsy device includes a first arm, a second arm spaced apart from the first arm, and a first probe unit. The first probe unit is mounted to the first arm and positioned such that, with the first and second arms coupled to the cranium, the first probe unit is positioned over and in contact with one of a left and right temporal region of the cranium. The first probe unit has a first transducer array configured to emit focused radiation at an ultrasonic frequency or frequency range and a first transcranial doppler probe moveable relative to the first transducer array and configured to emit sound waves.

### CROSS-REFERENCE TO RELATED APPLICATION

This is a continuation-in-part of U.S. patent application Ser. No. 17/923,274, filed Nov. 4, 2022, which is a U.S. national stage entry of International Application Serial No. PCT/US2021/045158, filed Aug. 9, 2021, which claims the benefit of and priority to U.S. Provisional Patent Application Ser. No. 63/064,008, filed Aug. 11, 2020, the disclosures of which are all expressly incorporated herein by reference in their entireties.

### TECHNICAL FIELD

The present disclosure relates generally to instruments and techniques utilizing ultrasound to fractionate clots resulting from acute ischemic stroke.

### BACKGROUND

Acute ischemic stroke (AIS) is the most common type of stroke, and Large Vessel Occlusion (LVO) is one of the most common types of AIS. For LVO, conventional treatments include intravenous thrombolytic medications (ITM) and/or invasive mechanical thrombectomy. Sonothrombolysis is a conventional therapy which uses focused ultrasound to fractionate clots or to increase the effectiveness of intravenous thrombolytic medication. Transcranial sonothrombolysis has been previously implemented in the form of an operator-independent helmet, and was used, along with ITM. Histotripsy is a subset of sonothrombolysis which employs lower frequency (1-MHz) pulses and fractionates soft tissue through controlled cavitation using focused, high-intensity ultrasound pulses.

### SUMMARY

The present disclosure may comprise one or more of the features recited in the attached claims, and/or one or more of the following features and combinations thereof. In one aspect, a histotripsy device may comprise a headset configured to be mounted to an about a cranium of a human or animal, at least one transducer mounted to the headset and positioned such that, with the headset mounted to and about the cranium, the at least one transducer is positioned over and in contact with at least one temporal or suboccipital region of the cranium, the at least one transducer configured to emit focused radiation at an ultrasonic frequency or frequency range, at least one processor, and at least one memory having instructions stored therein executable by the at least one processor to cause the at least one processor to activate the at least one transducer to produce at least one pulse of ultrasonic radiation having a pulse duration of 1 milliseconds or longer.

In another aspect, a histotripsy device may comprise a headset configured to be mounted to a cranium of a human or animal, at least one transducer array mounted to the headset and positioned such that, with the headset mounted to the cranium, the at least one transducer array is positioned over and in contact with at least one temporal or suboccipital region of the cranium, the at least one transducer array including multiple transducers or transducer segments each having a focal length of between about 70 to 165 mm and configured to emit focused radiation at an ultrasonic frequency or frequency range, and control circuitry responsive to a control signal input to drive the multiple transducers or transducer segments to produce pulsed ultrasonic radiation.

### BRIEF DESCRIPTION OF THE DRAWINGS

FIG. 1A is a right front perspective view of an embodiment of an operator-independent histotripsy device mounted to and about a cranium of a human.

FIG. 1B is a right rear perspective view of the device of FIG. 1A mounted to and about the cranium.

FIG. 1C is a left rear perspective view of the device of FIGS. 1A and 1B mounted to and about the cranium.

FIG. 2 is a simplified schematic diagram of an embodiment of the control module illustrated in FIG. 1A.

FIG. 3 is a simplified schematic diagram of an embodiment of one of the transducer arrays illustrated in FIGS. 1A-1C.

FIG. 4 is a perspective view of an embodiment of one of the transducers illustrated in FIG. 3.

FIG. 5 is a cross-sectional view of the transducer illustrated in FIG. 4 as viewed along section lines 5-5 thereof.

FIG. 6 is a perspective view of an instrument assembly including a histotripsy device configured to determine a location of a blood clot and treat the blood clot.

FIG. 7 is an enlarged view of the histotripsy device included in the instrument assembly of FIG. 6.

FIG. 8 is a perspective view of the histotripsy device of FIG. 7 coupled to a cranium of a human.

FIG. 9 is a cross-sectional view of a probe unit of the histotripsy device of FIG. 7.

FIG. 10 is a diagrammatic view of a transducer array included in the probe unit of FIG. 9.

FIG. 11 is a simplified schematic diagram of an embodiment of a control module of the instrument assembly of FIG. 6.

### DESCRIPTION OF THE ILLUSTRATIVE EMBODIMENTS

For the purposes of promoting an understanding of the principles of this disclosure, reference will now be made to a number of illustrative embodiments shown in the attached drawings and specific language will be used to describe the same.

This disclosure relates to a histotripsy device for fractionating clots resulting from acute ischemic stroke (AIS) and/or resulting from other events or conditions. The disclosed histotripsy device is portable and operator-independent, and illustratively includes a headset carrying at least one transducer array configured to emit and direct high-intensity, focused radiation, in the ultrasonic frequency range, into the cranium of a human or animal. The device further illustratively includes a control module operatively coupled to the headset for controlling operation of the one or more transducer arrays.

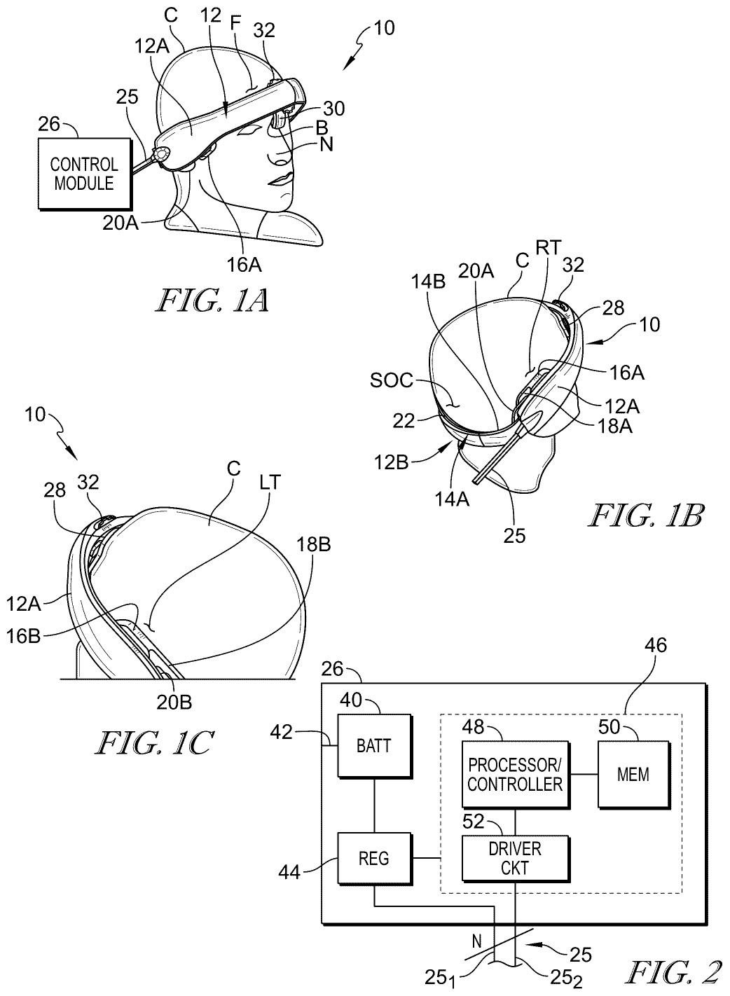

Referring now to FIGS. 1A-1C, an embodiment is shown of a histotripsy device 10 for fractionating clots resulting from AIS or other events/conditions. In the illustrated embodiment, the device 10 includes a head-mounted device or headset 12 illustratively provided in the form of a headband sized and configured to be mounted to and about the cranium, C, of a human (as depicted) or animal. In some alternate embodiments the headset 12 may be provided in other forms, examples of which may include, but are not limited to, a helmet, visor, or the like. In some embodiments, the headset 12 may, in any form, be configured to be mounted completely about the cranium, C, as depicted in FIGS. 1A-1C, although in alternate embodiments the headset 12 may be configured to extend only partially about the cranium C.

In the illustrated embodiment, the headset 12 illustratively includes a front band 12A and a rear band 12B configured to be operatively coupled thereto, wherein the combination of the front and rear bands 12A, 12B extend completely about the cranium C as illustrated in FIGS. 1A and 1B. The front band 12A is illustratively formed of a polymer, e.g., in the form of a rigid or semi-flexible plastic material, and is sized and configured to extend about a front portion of the cranium, C, e.g., generally in a C-shape, with opposed ends terminating approximately at or adjacent to each ear 20A, 20B on either side of the cranium C. In the illustrated embodiment, a pair of temporal transducer arrays 16A, 16B are mounted to interior surfaces of the front band 12A and positioned relative to the band 12A so as to be located generally over, and in contact with, the right and left temporal regions, RT and LT respectively, of the head (and brain), i.e., just anterior of each respective ear 20A, 20B. In some embodiments, as illustrated by example in FIGS. 1B and 1C, the transducer arrays 16A, 16B may each include a respective ear registration or locating member 18A, 18B. Illustratively, the ear locating members 18A, 18B are sized and configured to be positioned adjacent to a top (or anterior or posterior) portion of the auricles of the ears 20A, 20B so as to properly position the transducer arrays 16A, 16B over the temporal regions RT, LT of the head of the patient. In some embodiments, the ear locating members 18A, 18B may be sized and configured to be positioned between a portion of the auricles of the ears 20A, 20B, although in other embodiments the ear locating members 18A, 18B may be sized and configured to be positioned adjacent to the auricles. In some embodiments, the transducer arrays 16A, 16B are fixed in position relative to the front band 12A. In alternate embodiments, either or both of the transducer arrays 16A, 16B may be movable mounted to the front band 12A, and in such embodiments one or more conventional position adjustment structures may be incorporated into, or mounted to, the front band 12A to provide for axial and/or transverse adjustment of the position(s) of the transducer array(s) relative to the front band 12A.

The rear band 12B illustratively includes a transducer array carrier 14A and an elastic band 14B operatively coupled to the carrier 14A and to the terminal ends of the front band 12A. Illustratively, the elastic band 14B is sized to be stretchable to allow the headband 12 to accommodate, i.e., be mounted to and about, crania of different sizes. In some embodiments, one or more conventional adjustment members may be coupled to the elastic band 14B to provide for lengthening and/or shortening thereof. In any case, a suboccipital transducer array 22 is illustratively mounted to an inner surface of the transducer array carrier 14A and positioned relative to the rear band 12B so as to be located generally over, and in contact with, the suboccipital region, SOC, of the head of the patient, i.e., inferior to the occipital region of the cranium and above the level of the second cervical vertebra or, in other words, overlying the suboccipital triangle. In some embodiments, the transducer array 22 is fixed in position relative to the transducer array carrier 14A (and relative to the rear band 12B). In alternate embodiments, the transducer array 22 may be movable mounted to the transducer array carrier 14A, and therefore relative to the rear band 12B, and in such embodiments one or more conventional position adjustment structures may be incorporated into, or mounted to, the transducer array carrier 22 to provide for axial and/or transverse adjustment of the position of the transducer array relative to the transducer array carrier 22 and/or relative to the rear band 12B.

In one embodiment, each transducer array 16A, 16B, 22 illustratively includes an eight segment, spherically focused, 1 MHz transducer, having a 10 cm aperture and 7.5 cm focal length, and having an f-number greater than 0.9, e.g., between 1.5 and 1.75. It will be understood that in alternate embodiments, one or more, or all, of the transducer arrays 16A, 16B, 22 may include more or fewer transducer segments, may be configured to emit radiation at frequencies and/or frequency ranges greater or lesser than 1 MHz, have larger or smaller apertures, have greater or lesser focal lengths and/or have greater or lesser f-numbers. It will be further understood that alternate embodiments of the device 10 may include any number of temporal and/or suboccipital transducer arrays. It will also be understood that in some alternate embodiments, the device 10 may include only one or both of the temporal transducer arrays 16A, 16B, or only the suboccipital transducer array 22. It will be further still understood that in some alternate embodiments, the device 10 may include one or more transducer arrays suitably positioned relative to the headset 12 so as to be positioned over, and in contact with, any region(s) of the cranium (or brain), face and/or neck of the patient.

The headband 12 further illustratively includes an adjustment mechanism for tightening and loosening the headband 12 to and about the cranium C of the patient. In the illustrated embodiment, the adjustment mechanism is provided in the form of a forehead pad 28 operatively coupled to an adjustment wheel 32, with the pad 28 and the wheel 32 both operatively coupled to the headband 12 at a portion thereof positioned adjacent to the forehead F of the patient. The forehead pad 28 is sized and configured to contact a portion of the forehead, F, of the patient, and the adjustment wheel 32 is configured to cause, upon turning of the wheel 32, the forehead pad 28 to advance toward or retract from the surface of the forehead F so as to increase or decrease, respectively, the distance between the forehead pad 28 and the inner surface of the headband 12 to thereby tighten or loosen, respectively, the headband 12 to and about the cranium, C, of the patient. In some embodiments, as illustrated by example in FIG. 1A, the adjustment mechanism may further include a nose bride registration or locating device 30 coupled to the forehead pad 28 and configured to locate the pad 28 over the bridge, B, of the nose, N, of the patient.

In the illustrated embodiment, the histotripsy device 10 further includes a control module 26 operatively coupled to headband 12 via a cable 25. Illustratively, one end of the cable 25 is mechanically attached, i.e., affixed, to the front band 12A of the headband 12 as illustrated by example in FIGS. 1A and 1B, and the cable 25 also carries electrical conductors, e.g., wires, one or more of which is operatively coupled to each of the transducer arrays 16A, 16B, 22. In some embodiments, the cable 25 is fixed to and between the headband 12 and the control module 26, e.g., to a housing of the control module 26. In alternate embodiments, the cable 25 and the headband 12 and/or the cable 25 and the control module 26 may be fitted with one or more suitable connectors for electrically connecting the cable 25 to the headband 12 and/or to the control module 26.

Referring now to FIG. 2, a simplified example is shown of an embodiment of the control module 26. In the illustrated embodiment, the control module 26 includes at least one battery 40 (and/or other source of electrical power). In some embodiments, the battery(s) 40 is/are rechargeable, and in such embodiments the module 26 further includes a charging port 42 electrically coupled to the battery(s) 40 and configured to operatively connect to a conventional charging cable connectable to an external power source. In any case, the module 26 further illustratively includes regulator circuitry 44 electrically coupled to the battery(s) 40. The regulator circuitry 44 is conventional and includes one or more regulator circuits configured to convert the voltage of the battery 40 to one or more voltages suitable to power control circuitry 46 on-board the module 26 and to power the transducer arrays 16A, 16B, 22. In this regard, the regulator circuitry 44 has at least one output electrically coupled to the control circuitry 46 and at least one output coupled (or configured to be coupled) to the headband 12 via the cable 25.

In the illustrated embodiment, the control circuitry 46 includes at least one processor or controller 48 operatively coupled to (or integral with) at least one memory device 50, and operatively coupled to driver circuitry 52. It will be understood that the terms “processor” and “controller” used in this disclosure is comprehensive of any computer, processor, microchip processor, integrated circuit, or any other element(s), whether singly or in multiple parts, capable of carrying programming for performing the functions specified in the claims and this written description. In this regard, the at least one processor or controller 48 may be a single such element which is resident on a printed circuit board with the other elements of the control circuitry 46, or may be or include two or more elements resident with the other elements of the control circuitry 46 and/or resident in one or more locations of the headband 12. The memory 50 is likewise conventional and includes instructions stored therein which are executable by the processor or controller 48 to carry out the various functions of the control module 26 described herein. The driver circuitry 52 is conventional and includes one or more driver circuits configured to drive, i.e., actuate, the transducer arrays 16A, 16B, 22. In this regard, the driver circuitry 52 has at least one output electrically coupled (or configured to be coupled) to the headband 12 via the cable 25.

In some alternate embodiments, headset 12 may carry its own battery and driver circuitry for powering and actuating the transducer arrays 16A, 16B, 22. In some such embodiments, the control module 26 and the headband 12 may each include conventional circuitry configured for wireless communication with one another, and the processor(s) or controller(s) 48 may in such embodiments thereby wirelessly control operation of the transducer arrays 16A, 16B, 22.

As briefly described above in the BACKGROUND section, histotripsy is a subset of sonothrombolysis which employs relatively lower frequency pulses to fractionate soft tissue through controlled cavitation. Generally, there are two different mechanisms of cavitation in histotripsy: (1) shock-scattering, and (2) intrinsic threshold (also known as microtripsy). Shock-scattering employs short-duration, high-amplitude pulses of ultrasound with multiple positive and negative half-cycles that interact to produce cavitation clouds, sometime called “bubble clouds,” which mechanically break down clot tissue. Intrinsic threshold histotripsy, on the other hand, employs pulses with a single large tensile phase to produce bubble clouds. These two mechanisms of cavitation thus utilize different pulse durations and different peak negative pulse pressures.

As briefly described above, the memory 50 illustratively includes instructions stored therein which are executable by the processor or controller 48 to carry out the various functions of the control module 26. Illustratively, the instructions stored in the memory 50 include instructions to control the transducers 16A, 16B and 22 to produce 1 MHz fundamental frequency pulses to nucleate bubble activity through either or both of the intrinsic threshold and shock-scattering mechanisms described above. In an embodiment of the former case, i.e., intrinsic threshold, the instructions stored in the memory 50 include instructions executable by the processor(s) or controller(s) 48 to cause the processor(s) or controller(s) 48 to control one or more of the transducers 16A, 16B to produce 1 MHz fundamental frequency pulses of 1 millisecond (ms) in duration (although frequency pulse durations outside of this range are contemplated). In an embodiment of the latter case, i.e., shock-scattering, the instructions stored in the memory 50 include instructions executable by the processor(s) or controller(s) 48 to cause the processor(s) or controller(s) 48 to control one or more of the transducers 16A, 16B to produce 1 MHz fundamental frequency pulses of 5 ms (although frequency pulse durations outside of this range are contemplated). In either case, the histotripsy pulses will be generated by the transducers 16A, 16B, 22 with pulse durations of 1 MHz or longer. The histotripsy pulses will have a single tensile phase in excess of 35 mega-Pascals (MPa) (e.g., in the range of approximately 35 to 40 MPa, although pressures outside of this range are contemplated), and the peak negative pressure of the pulses will be between approximately 20 and 30 MPa (although pressures outside of this range are contemplated). In one embodiment, at each location, i.e., with each transducer 16A, 16B, 22, the transducer 16A, 16B, 22 will be controlled by the processor(s) or controller(s) 48 to generate, and apply to the respective region, between 500 and 1000 pulses at a >40 Hz rate for a total insonation time of between 20 and 60 seconds, although in alternate embodiments more or fewer pulses may be applied and/or may be applied at 40 Hz or less, for any desired total insonation time.

The histotripsy device 10, operated as described above, is capable of fractionating clots without adjunctive intravenous thrombolytic medications (ITM). Advantageously, because the device 10 is capable of therapy independent of ITM, it will allow patients to avoid the risks and side-effects of such medications. In alternate embodiments, however, it is to be understood that the histotripsy device 10 may also be used in conjunction with ITM.

The Histrotripsy device 10 is compact, portable and is operator-independent, and it can therefore be implemented in a wide-variety of clinical settings. For example, it is estimated that approximately 2 million neurons per minute are lost during an acute ischemic stroke (AIS), and AIS treatment is accordingly extremely time-dependent. In this regard, because the Histotripsy device 10 will be fast-acting and easy to use, it can be integrated into an emergency room setting, which could greatly reduce time-to-treatment and thereby potentially improve treatment outcomes. Moreover, because the Histotripsy device 10 is relatively small (e.g., headband or helmet-sized) and operator-independent, it could further be integrated into rural hospitals and mobile stroke units. Use of the Histotripsy device 10 by first responders (e.g., EMT's) or emergency room clinicians at rural hospitals, for example, can thus provide treatment to AIS victims far sooner than they would otherwise receive using conventional therapies.

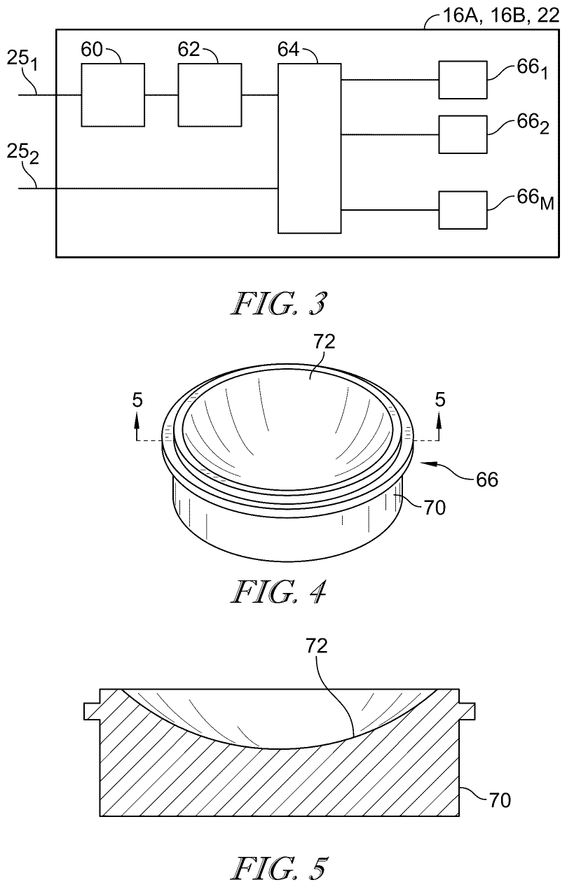

Referring now to FIG. 3, a schematic diagram is shown of an embodiment of one of the transducer arrays 16A, 16B, 22. In one embodiment, the transducer arrays 16A, 16B, 22 are configured identically as illustrated in FIG. 3, although in alternate embodiments one or more of the transducer arrays 16A, 16B, 22 may be configured differently than the others, e.g., by including more or fewer transducers, by having differently configured transducers, i.e., having different operating characteristics, and/or by including more, fewer or different on-board control circuits. In the embodiment illustrated in FIG. 3, the electrical power signal line 25₁ exiting the control module 26 of FIG. 2 is electrically connected to the input of a high-voltage power supply circuit 60, and the output of the supply 60 is electrically connected to an input of an energy storage circuit 62. In one embodiment, the energy storage circuit is implemented in the form of a capacitor bank, in which case the power supply 60 may illustratively be designed for capacitor charging, although in alternate embodiments one or more other or additional energy storage circuits may be used. The output of the energy storage circuit 62 is electrically connected to a power supply input of a multi-channel output circuit 64 having a number, M, of outputs each electrically connected to the input of a different one of a corresponding number, M, of transducers (or transducer segments) 66₁-66_{M}, where M may be any positive integer. In one embodiment, as described above, M=8, although in alternate embodiments M may be greater or less than 8. In the illustrated embodiment, the multiple output stages of the multi-channel output circuit 64 include transducer matching networks for matching the electrical input characteristics of the transducers 66₁-66_{M}. In any case, the control signal line 25₂ exiting the control module 26 of FIG. 2 is electrically connected to a control signal input of the multi-channel output circuit 64.

In the embodiment illustrated in FIG. 2, the operating frequency of the transducers 66₁-66_{M} is generated by the processor/control circuit 48 and the driver circuitry 52 of the control module 26, illustratively in the form of a low-voltage, i.e., low-power, square wave control signal, and this low-voltage square wave control signal is then fed to the transducer array 16A, 16B, 22 depicted by example in FIG. 3. In the embodiment illustrated in FIG. 3, the control circuitry 60, 62 and 64 is illustratively configured as a high-voltage switch mode pulse generator which converts the low-voltage square wave control signal supplied by the control module 26 to a high-voltage, i.e., high-power, square wave drive signal for driving the transducers 66₁-66_{M}. The energy for each output pulse produced by the output circuit 64, and supplied as an input drive signal to a respective one of the transducers 66₁-66_{M}, is stored in the energy storage circuit 62, which is recharged between pulses by the high-voltage supply circuit 60. Based on the low-voltage square wave control signal supplied by the control module 26 and on the high-voltage energy stored in the energy storage circuit 62, the multi-channel output circuit 64 generates and supplies to each of the transducers 66₁-66_{M} a high-voltage, square wave drive signal.

In one example embodiment, the control module 26, depicted by example in FIG. 2, and the control circuitry 60, 62, 64 of the transducer array 16A, 16B, 22, depicted by example in FIG. 3, together produce the high-voltage, square wave drive signals with the following features: signal frequency 1.5 MHz+/−50 kHz, output voltage 0-500 V_{PK}, output current 10 A per channel, pulse repetition rate 1-100 Hz, and burst length 1-30 cycles. It will be understood that in alternate embodiments, one or more of the foregoing values may be greater or lesser.

Referring now to FIGS. 4 and 5, an example embodiment is shown of one of the transducers 66, depicted in schematic form in FIG. 3, is shown. The transducer 66 illustratively has a base 70 and a transducer head 72 mounted to the base. The ultrasound-emitting surface of the transducer head 72 is illustratively concave. In the illustrated embodiment, the base 70 is generally cylindrical in lateral cross-sectional shape, as is the transducer head 72, and the concave, ultrasound-emitting surface of the transducer head 72 is accordingly a truncated sphere as best shown in FIG. 5. In alternate embodiments, the base and/or the transducer head 72 may have a non-cylindrical, lateral cross-sectional shape, and/or the ultrasound-emitting surface of the transducer head 72 may be non-concave, e.g., planar, convex or other shape.

In the illustrated embodiment, the transducer 66 is self-focusing and configured to produce ultrasonic pulses with a center frequency of approximately 1.5 MHz, although in alternate embodiments the transducer 66 may not self-focusing and/or may be configured to produce ultrasonic pulses with a center frequency greater or less than 1.5 MHz. In one example embodiment, the spherical-geometry transducer 66 depicted by example in FIGS. 4 and 5 has the following structural features: 100 mm diameter, focal length=75 mm, and the following ultrasonic pulse signal features: 1.5 MHz+/−50 kHz center frequency, minimum focal tensile pressure=−35 MPa, maximum surface pressure amplitude=300 kPa and linear focal gain=120. It will be understood that in alternate embodiments, one or more of the foregoing values may be greater or lesser. In some embodiments, one or more of the transducers 66₁-66_{M} may have the above geometry and features, whereas others of the transducers 66₁-66_{M} (and/or one or more of the transducers 66₁-66_{M} in one or more other transducer arrays) may have different structural and/or pulse signal features, e.g., focal length=163 mm, minimum focal tensile pressure=−25 MPa, maximum surface pressure amplitude=500 kPa and linear focal gain=49. In some alternate embodiments, one or more of the transducers 66₁-66_{M} (and/or one or more of the transducers 66₁-66_{M} in one or more other transducer arrays) may a focal length anywhere within the range of between approximately 70 mm and 165 mm.

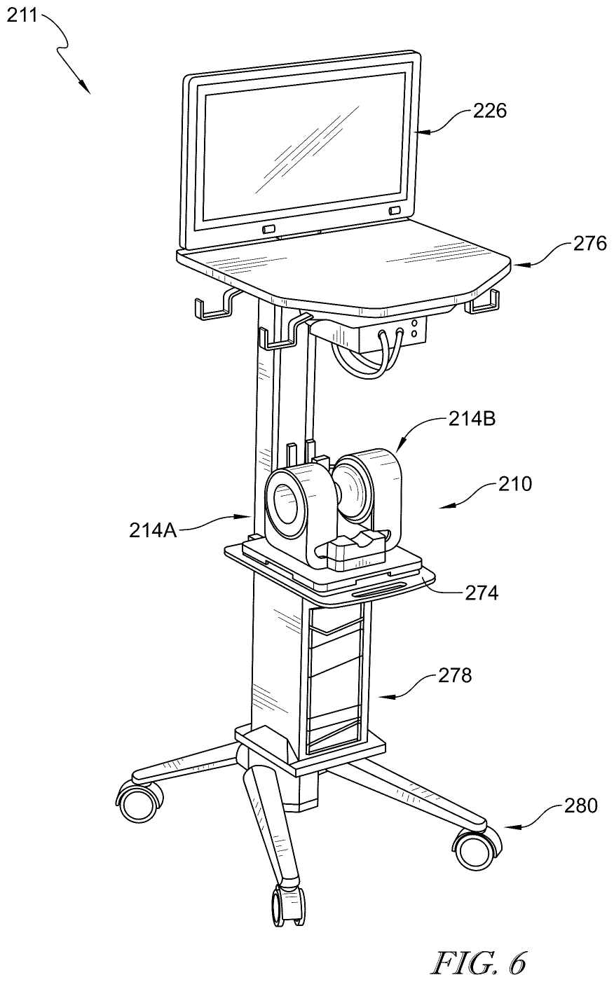

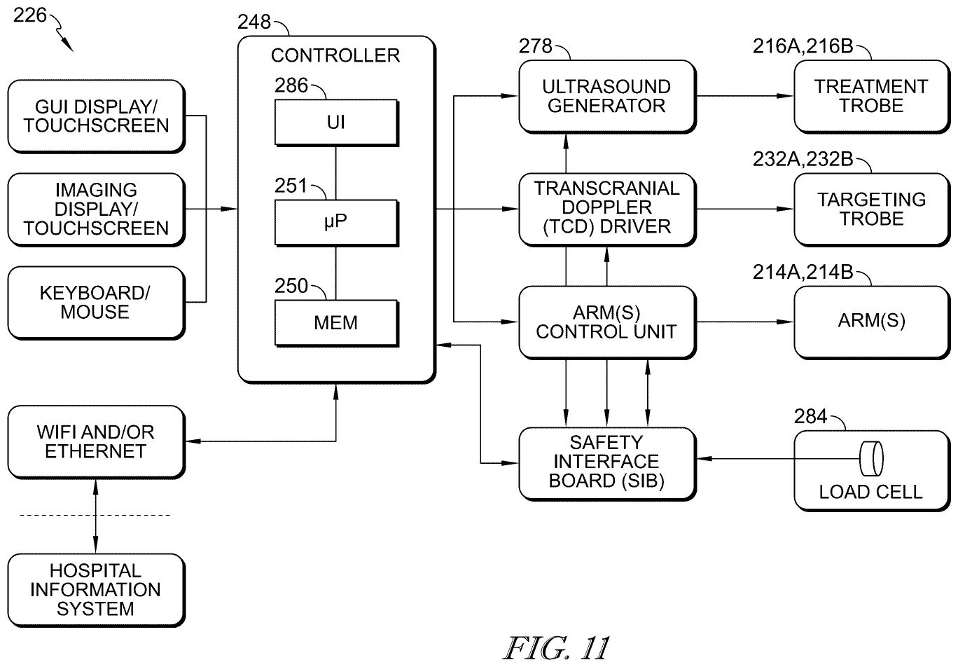

Another histotripsy device 210 is disclosed herein, as shown in FIGS. 6-11. The histotripsy device 210 is substantially similar to the histotripsy device 10 shown in FIGS. 1-5 and described herein. Accordingly, similar reference numbers in the 200 series indicate features that are common between the histotripsy device 210 and the histotripsy device 10. The description of the histotripsy device 10 is incorporated by reference to apply to the histotripsy device 210, except in instances when it conflicts with the specific description and the drawings of the histotripsy device 210.

In the illustrated embodiment, the histotripsy device 210 is included in an instrument assembly 211, as shown in FIG. 6. The instrument assembly 211 includes a device tray 274, a computer tray 276, a generator 278, and/or a plurality of wheels 280. The device tray 274 stores the histotripsy device 210 thereon. The computer tray 276 is located above the device tray 274 and houses a control module 226 thereon. In some embodiments, the control module 226 may be embodied as a computer. The generator 278 provides power to the histotripsy device 210 during use of the histotripsy device 210. The plurality of wheels 280 allows the instrument assembly 211 to be easily moved between locations. It should be appreciated that the histotripsy device 210 may be provided without the other components of the instrument assembly 211.

Though described as a histotripsy device 210, the device 210 may use any ultrasound modality. For example, the device 210 may be a histotripsy device, a high-intensity focused ultrasound device using high-intensity focused ultrasound, a low-intensity focused ultrasound device using low-intensity focused ultrasound, a burst-wave lithotripsy device, a phased beamforming device, a continuous wave device, a coded pulse device, any other suitable device, or any suitable combination of the same.

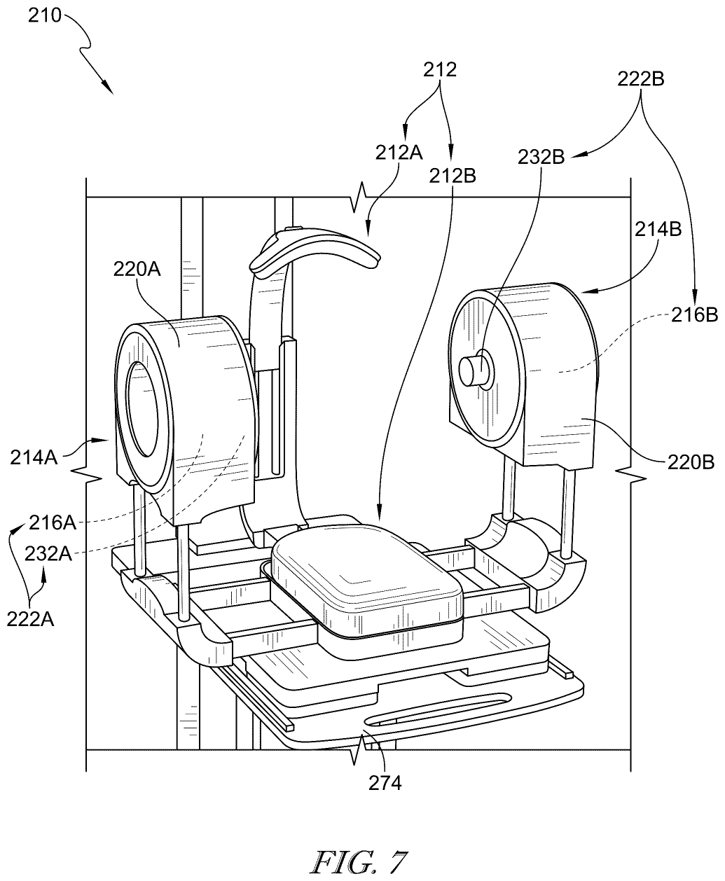

The computer tray 276 can be raised and lowered relative to the device tray 274 between a storage mode (not shown) and a use mode, as shown in FIG. 6. The device tray 274 can be moved between a storage mode, as shown in FIG. 6, and a use mode, as shown in FIG. 7. As shown in FIG. 7, the device tray 274 is pulled outwardly.

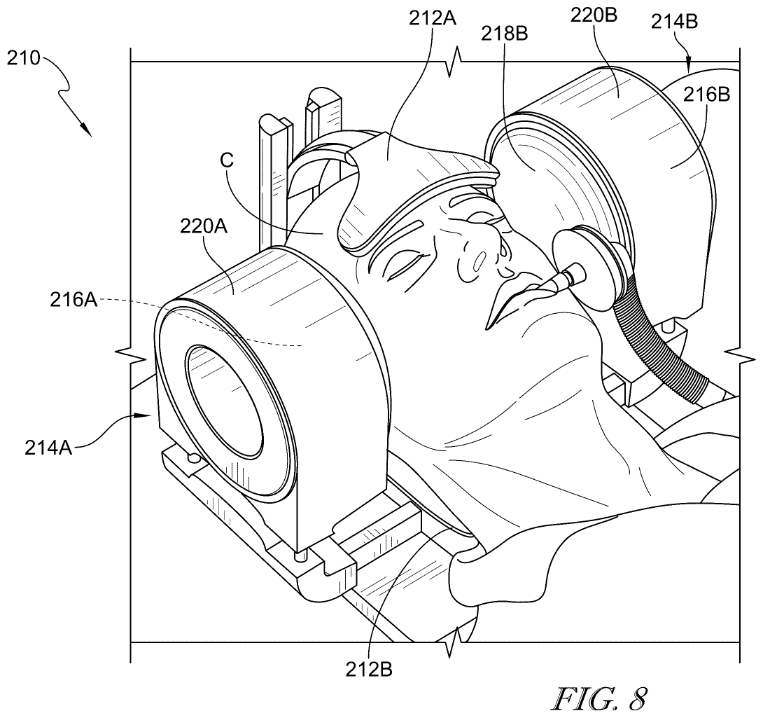

The histotripsy device 210 includes a headset 212, a first arm 214A, and a second arm 214B spaced apart from the first arm 214A, as shown in FIG. 7. The headset 212 includes a head stabilizer 212A and a headrest 212B. During use, as shown in FIG. 8, the headrest 212B supports a posterior portion of the cranium C of a human (as depicted) or an animal and the head stabilizer 212A supports an anterior portion of the cranium C. The first arm 214A engages one of the ears of the patient, and the second arm 214B engages another one of the ears of the patient.

Though shown and described as including a headset 212 and two arms 214A, 214B, the histotripsy device 210 may include any wearable configuration, such as, but not limited to, a helmet, a band, a cap, a collar, a gantry-mounted array, a robotic frame, or a fixed bedside mount.

As shown in FIGS. 6 and 7, the head stabilizer 212A is movable upwardly and downwardly relative to the headrest 212B. In this way, the head stabilizer 212A can be moved upwardly while the cranium C is being positioned on the headrest 212B, and once positioned, the head stabilizer 212A can be moved downwardly until snuggly fit onto the cranium C.

The arms 214A, 214B are moveable toward and away from one another, as shown in FIGS. 6 and 7. This allows the arms 214A, 214B to be compactly stored (see FIG. 6) and to fit on various different cranium sizes (see FIG. 8). As shown in FIGS. 7 and 8, the arms 214A, 214B can be moved away from one another while the cranium C is being positioned on the headrest 212B, and once positioned, the arms 214A, 214B can be moved toward one another until snuggly fit onto the cranium C.

Each of the arms 214A, 214B has a base 220A, 220B, an earpad 218A, 218B coupled to the corresponding base 220A, 220B, and a probe unit 222A, 222B. The probe units 222A, 222B are coupled to the corresponding base 220A, 220B. Each of the earpads 218A, 218B is coupled to the corresponding base 220A, 220B to locate the probe units 222A, 222B at least partially between the earpads 218A, 218B and the base 220A, 220B. It should be appreciated that the earpads 218A, 218B are removed in FIG. 7 for clarity of description.

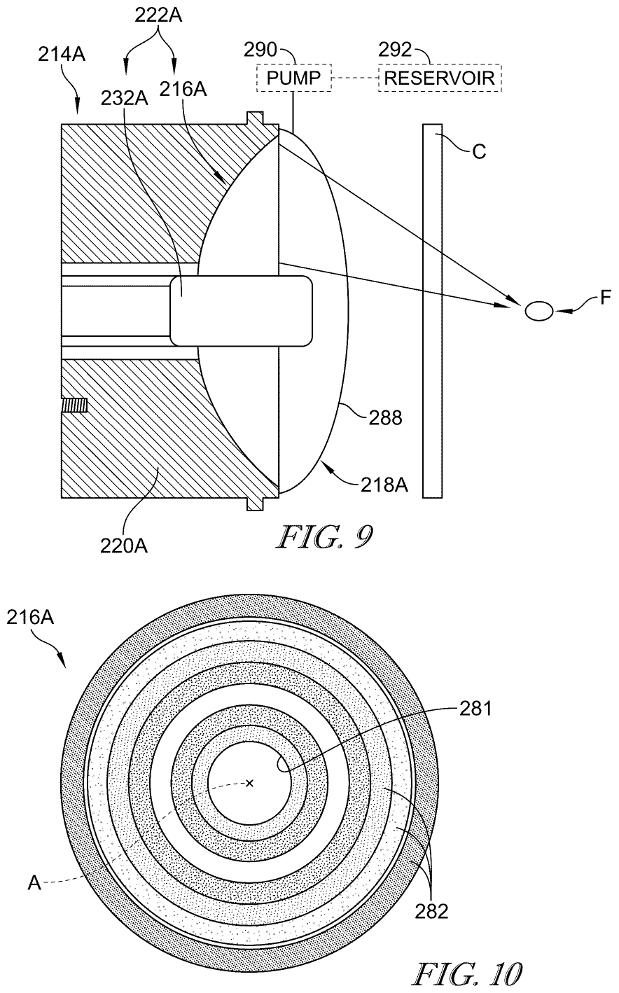

The earpads 218A, 218B are mounted to the arms 214A, 214B so as to be located generally over, and in contact with, the right and left temporal regions, respectively, of the cranium C. In this way, the probe units 222A, 222B are located generally over the right and left temporal regions of the cranium C. Each of the probe units 222A, 222B includes a transducer array 216A, 216B and a transcranial doppler probe 232A, 232B, as shown in FIGS. 7 and 9. One or more of the transducer arrays 216A, 216B are configured to emit focused radiation at an ultrasonic frequency or frequency range. One or more of the transcranial doppler probes 232A, 232B are configured to emit sound waves.

In some embodiments, the histotripsy device 210 includes alternative imaging or feedback modalities instead of or in addition to the transcranial doppler probes 232A, 232B to detect and/or confirm the location of the blood clot. For example, the histotripsy device 210 may include focused ultrasound (fUS) probes, computed tomography (CT) imaging, magnetic resonance imaging (MRI), electroencephalogram (EEG) feedback, acoustic emissions monitoring, thermal mapping, and/or a machine learning interference without imaging.

Each of the transducer arrays 216A, 216B defines a central through hole 281, as shown in FIGS. 9 and 10. The central through hole 281 extends through the respective transducer array 216A, 216B along a central axis A thereof. Each of the transcranial doppler probes 232A, 232B is received in the central through hole 281 of the corresponding transducer array 216A, 216B. The transcranial doppler probes 232A, 232B are moveable relative to the transducer arrays 216A, 216B along the central axis A through the central through hole 281. In illustrative embodiments, each of the transcranial doppler probes 232A, 232B has six degrees of freedom. In this way, the transcranial doppler probes 232A, 232B can move axially along the central axis A and radially relative to the central axis A (once sufficiently clear from the central through hole 281). In alternative embodiments, the transcranial doppler probes 232A, 232B can move axially along the central axis A and each of the arms 214A, 214B has six degrees of freedom.

In some embodiments, the transcranial doppler probes 232A, 232B include a linear actuator, a drive screw, a pneumatic system, or any other system capable of moving the transcranial doppler probes 232A, 232B.

As shown in FIG. 10, the transducer array 216A includes a plurality of annular segments 282, each of which extends around the central axis A. Each of the plurality of annular segments 282 is at a different radial distance relative to the central axis A. In illustrative embodiments, the plurality of annular segments 282 includes seven annular segments 282. It will be understood that each of the transducer arrays 216A, 216B are the same. Thus, the description of the transducer array 216A and FIG. 10 applies to the transducer array 216B.

In the illustrated embodiment, the control module 226 includes a controller 248 operatively connected to the probe units 222A, 222B. In some embodiments, the controller 248 and/or the generator 278 are/is coupled to the probe units 222A, 222B via a cable (not shown). In some embodiments, the controller 248 and/or the generator 278 wirelessly communicate with the probe units 222A, 222B. The controller 248 includes at least one processor 251 operatively coupled to (or integral with) at least one memory device 250. The memory 250 includes instructions stored therein which are executable by the processor 251 to carry out the various functions of the controller 248 described herein.

During use, the histotripsy device 210 is removed from the device tray 274 and placed on a stretcher or a bed. The arms 214A, 214B are moved away from one another, and the head stabilizer 212A is moved upwardly relative to the headrest 212B. The cranium C of an incubated and sedated patient is then positioned on the headrest 212B. The head stabilizer 212A is then moved downwardly into contact with the cranium C, and the arms 214A, 214B are moved toward one another into contact with the cranium C, as shown in FIG. 8.

In some embodiments, the movement of the head stabilizer 212A and the arms 214A, 214B is manually performed by an operator. In some embodiments, the movement of the head stabilizer 212A and the arms 214A, 214B is automatically performed via the arms 214A, 214B. For example, one or more sensors 284 may be coupled to the headrest 212B and/or the arms 214A, 214B to detect pressure and/or force between the headrest 212B and/or the arms 214A, 214B and the cranium C. As another example, one or more sensors 284 (e.g., optical sensors) may detect landmark registration of the cranium C (e.g., ear(s)) for optical or anatomical positioning of the arms 214A, 214B. The movement of the head stabilizer 212A and the arms 214A, 214B is automatically performed based on inputs from the one or more sensors 284. The one or more sensors 284 may be pressure sensors, pressure transducers, load cells, force sensors, or any other suitable type of sensor.

The at least one sensor 284 coupled to the arms 214A, 214B provides inputs to the processor 251 indicative of a pressure between the respective earpad 218A, 218B and the cranium C. In some embodiments, based on the inputs, the processor 251 is configured to move one or more of the arms 214A, 214B to tighten or loosen the arms 214A, 214B relative to the cranium C. In some embodiments, the inputs are provided to the operator such that the operator can manually move the arms 214A, 214B once the input is received.

Once properly fit to the cranium C, one or more of the transcranial doppler probes 232A, 232B may be activated to produce the sound waves to determine a location of a blood clot. As an example, the operator may activate the one or more of the transcranial doppler probes 232A, 232B by pressing a button, such as a ‘SCAN’ button, on a user interface 286 of the controller 248. While activated and producing the sound waves, the one or more of the transcranial doppler probes 232A, 232B can move relative to the transducer arrays 216A, 216B, as described above. The one or more of the transcranial doppler probes 232A, 232B use the sound waves to measure blood flow in blood vessels of the brain. In this way, the one or more of the transcranial doppler probes 232A, 232B can determine the location of the blood clot. The one or more of the transcranial doppler probes 232A, 232B send inputs to the processor 251. The processor 251, based on the inputs, determines the location of the blood clot, and then communicates the location to one or more of the transducer arrays 216A, 216B.

The one or more of the transducer arrays 216A, 216B may then be activated to produce at least one pulse of ultrasonic radiation to disintegrate and/or treat the blood clot. As an example, the operator may activate the one or more of the transducer arrays 216A, 216B by pressing a button, such as a ‘TREAT’ button, on the user interface 286 of the controller 248. Because the transducer arrays 216A, 216B and the transcranial doppler probes 232A, 232B are integrated into the same probe units 222A, 222B, the cranium C of the patient does not move between the locating of the blood clot and the disintegrating and/or treating of the blood clot. In traditional systems, the patient may have to move between different instruments and/or machines, which decreases the accuracy of the treatment of the blood clot.

The at least one pulse of ultrasonic radiation treats the blood clot by reducing its size, such as by disintegrating it, disassembling it, or breaking it up into at least two or more pieces, softening the blood clot, liquifying the blood clot, perforating the blood clot, inducing porosity into the blood clot, augmenting flow through the blood clot, partial recanalization of the blood clot, mechanical destabilization of the blood clot, and/or biological facilitation of clot degradation via pressure created by the at least one pulse of ultrasonic radiation.

In illustrative embodiments, the at least one memory device 250 has instructions stored therein executable by the at least one processor 251 to cause the at least one processor 251 to sequentially activate each of the plurality of annular segments 282 included in the first transducer array 216A and/or the second transducer array 216B to sequentially produce at least one pulse of ultrasonic radiation from each of the plurality of annular segments 282.

In some embodiments, each of the plurality of annular segments 282 is sequentially activated. For example, the outermost annular segment 282 may be activated first, then the second-most outermost annular segment 282 may be activated second, then the third-most outermost annular segment 282 may be activated third, etc. As another example, the innermost annular segment 282 may be activated first, then the second-most innermost annular segment 282 may be activated second, then the third-most innermost annular segment 282 may be activated third, etc. In another example, the annular segments 282 may be randomly activated such that a pattern (e.g., outer to inner or inner to outer) is not followed. In another example, the annular segments 282 may be simultaneously activated.

As shown in FIG. 9, the geometric path from each of the annular segments 282 to a focal point F is a constant distance (i.e., 75 millimeters). However, the path from one annular segment 282 to the focal point F through the different media (i.e., the bone of the cranium C) takes different length paths through the media (i.e., the bone of the cranium C) because of the angle. For example, the path through the cranium C from the outer-most annular segment 282 is longer than the path through the cranium C from the inner-most annular segment 282, as shown by the arrows in FIG. 9. As a result, a time delay in the ultrasound pulses occurs that needs to be compensated for so that the ultrasound pulse from each of the annular segments 282 reaches the focal point F at the same time to create maximum negative peak pressure. A phase delay (i.e., the sequential activation) accounts for the time delay through the cranium C. Thus, to ensure that the ultrasonic radiation from each of the plurality of annular segments 282 reaches the focal point F at the same time, the ultrasonic radiation from each of the plurality of annular segments 282 is activated at different times.

In some embodiments, the phases may also be adjusted to move the focal point F along the axis A. This allows the system to make a small adjustment to the focus position to compensate for clot breakup, to allow the distribution of cavitation over a larger occlusion, and/or to make minor adjustments to the energy focus without having to make mechanical adjustments to the probe units 222A, 222B. In some embodiments, the phases can be adjusted based on physiological conditions of the specific patient (such as cranium C thickness, race, ethnicity, gender, anatomy, age, vessel depth, clot composition, real-time feedback, etc.).

In some embodiments, the transducer arrays 216A, 216B may be activated based, at least in part, on the location of the blood clot. For example, inputs from the transcranial doppler probes 232A, 232B related to the location of the blood clot may be used by the controller 248 to modify a focal point of the transducer arrays 216A, 216B, to determine operating parameters of the transducer arrays 216A, 216B, etc. In some embodiments, the transducer arrays 216A, 216B may be aligned with the cranium C based on the location of the blood clot. For example, based on the location of the blood clot, the arms 214A, 214B may move relative to the cranium C to properly align the transducer arrays 216A, 216B with the blood clot.

In some embodiments, the histotripsy device 210 allows for tele-operation or remote-controlled targeting. In this way, the histotripsy device 210 may be used outside of hospital or medical settings.

In some embodiments, mixed frequency therapy may be provided by the transducer arrays 216A, 216B. The mixed frequency therapy uses lower frequency pulses (e.g., 0.75 Mhz) to deliver the bulk energy and higher frequency pulses (e.g., 1.0 Mhz) to get the desired small treatment volume. Higher frequency pulses (e.g., 1.0 Mhz) provides a small focusing volume that is appropriate for the rarefaction of a large vessel occlusion (i.e., clot) in the mid cerebral artery. However, the same higher frequency is absorbed in the skull bone interface (attenuation loss of around 75%). In the same condition, a 0.75 Mhz pulse transmission attenuation loss through skull bone is only 64.5%. Increasing the pulse amplitude at 1.0 Mhz increases the risk of tissue heating. Thus, using two (or more) transducer arrays 216A, 216B distributes the energy delivery path to reduce tissue heating due to attenuation loss.

Using the 0.75 Mhz frequency pulses allows the transmission of more ultrasound energy with less loss. However, the pressure delivered is not enough to create cavitation. Using the 1.0 Mhz frequency pulses allows the delivery of enough pressure to create cavitation, but in the smaller focal area that the 1 Mhz frequency allows. Both pulse delivery can be coordinated to allow delivery within the first few pulses, otherwise the periodic difference will start to cancel each other.

In such an embodiment, one of the transducer arrays 216A, 216B uses the 0.75 MHz pulses, and the other of the transducer arrays 216A, 216B uses the 1.0 MHz pulses. In alternative embodiments, both of the transducer arrays 216A, 216B uses the 0.75 MHz pulses, or both of the transducer arrays 216A, 216B uses the 1.0 MHz pulses.

In some embodiments, each transducer array 216A, 216B illustratively includes a seven segment, spherically focused, 1 MHz transducer, having a 10 centimeter aperture and 7.5 centimeter focal length, and having an f-number greater than 0.9, e.g., between 1.5 and 1.75. It will be understood that in alternate embodiments, one or more, or all, of the transducer arrays 216A, 216B may include more or fewer transducer segments, may be configured to emit radiation at frequencies and/or frequency ranges greater or lesser than 1 MHz, have larger or smaller apertures, have greater or lesser focal lengths and/or have greater or lesser f-numbers. It will also be understood that in some alternate embodiments, the device 210 may include only one or both of the temporal transducer arrays 216A, 216B, and/or the device 210 may include a suboccipital transducer array.

As shown in FIGS. 8 and 9, the earpads 218A, 218B each form an acoustic coupling layer. The acoustic coupling layer allows for an efficient transfer of acoustic energy from the transcranial doppler probes 232A, 232B and into the cranium C. The acoustic coupling layer matches the acoustic impedance between the patient's skin/tissue layer and the surface of the transcranial doppler probes 232A, 232B.

In some embodiments, the earpads 218A, 218B each include an outer wall 288 defining a hollow cavity therein. The hollow cavity is filled with a gel. The gel may be a polyacrylamide hydrogel, a polyvinyl alcohol hydrogel, a gelatin-based gel, a silicone-hydrogel, or any other suitable gel.

In some embodiments, as shown in FIG. 9, the histotripsy device 210 further includes a pump 290 and a reservoir 292. The reservoir 292 stores the gel therein, and the pump 290 directs the gel between the reservoir 292 and the hollow cavity of the earpads 218A, 218B. In this way, the volume of the earpads 218A, 218B can be adjusted such that the pressure between the earpads 218A, 218B and the cranium C is adjusted. The adjustable volume allows the air between the transcranial doppler probes 232A, 232B and the cranium C to be displaced. If air is present in the form of a layer or bubbles, the difference in the acoustic impedance causes the ultrasound energy to deflect or be absorbed when the ultrasound energy encounters the layer between the air and the acoustic coupling layer. Additionally, the adjustable volume allows for the surface of the earpads 218A, 218B and the surface of the cranium C to conform to one another.

In some embodiments, the controller 248 is in communication with the pump 290 to control the pump 290 to direct the gel into or out of the earpads 218A, 218B from or to the reservoir 292. For example, based on inputs from the at least one sensor 284, the controller 248 may operate the pump 290.

As briefly described above, the memory 250 illustratively includes instructions stored therein which are executable by the processor or controller 248 to carry out the various functions of the control module 226. Illustratively, the instructions stored in the memory 250 include instructions to control the transducer arrays 216A, 216B to produce 1 MHz fundamental frequency pulses to nucleate bubble activity through either or both of the intrinsic threshold and shock-scattering mechanisms described above. In an embodiment of the former case, i.e., intrinsic threshold, the instructions stored in the memory 250 include instructions executable by the processor(s) or controller(s) 248 to cause the processor(s) or controller(s) 248 to control one or more of the transducer arrays 216A, 216B to produce 1 MHz fundamental frequency pulses of 1 millisecond (ms) in duration (although frequency pulse durations outside of this range are contemplated). In an embodiment of the latter case, i.e., shock-scattering, the instructions stored in the memory 250 include instructions executable by the processor(s) or controller(s) 248 to cause the processor(s) or controller(s) 248 to control one or more of the transducer arrays 216A, 216B to produce 1 MHz fundamental frequency pulses of 5 ms (although frequency pulse durations outside of this range are contemplated). In either case, the histotripsy pulses will be generated by the transducer arrays 216A, 216B with pulse durations of 1 MHz or longer. The histotripsy pulses will have a single tensile phase in excess of 35 mega-Pascals (MPa) (e.g., in the range of approximately 35 to 40 MPa, although pressures outside of this range are contemplated), and the peak negative pressure of the pulses will be between approximately 20 and 30 MPa (although pressures outside of this range are contemplated). In one embodiment, at each location, i.e., with each transducer array 216A, 216B, the transducer arrays 216A, 216B, will be controlled by the processor(s) or controller(s) 248 to generate, and apply to the respective region, between 500 and 1000 pulses at a >40 Hz rate for a total insonation time of between 20 and 60 seconds, although in alternate embodiments more or fewer pulses may be applied and/or may be applied at 40 Hz or less, for any desired total insonation time.

In some embodiments, pulse compression may be used with the transcranial doppler probes 232A, 232B for resolution improvement. Transcranial doppler axial resolution accuracy is limited by the sample volume gate which can be on the order of 2 to 5 millimeters. Pulse compression is a method of increasing axial resolution in transcranial doppler ultrasound. Pulse compression is a signal processing technique that uses long ultrasound pulses for a strong echo but achieves better axial depth resolution of a short pulse. The method accomplishes this by coding the transmitted pulse (in frequency or phase) then compressing it in the time domain after receiving the signal using a matched filter (correlation).

Using pulse compression on the transcranial doppler probes 232A, 232B provides a compact and cost-effective method of targeting the large vessel occlusion in the mid-cerebral artery. The chirp coded pulses are a linear modulated (in an example, 1 to 1.3 Mhz) waveform sweep over time. The phase coded pulses (i.e., Barker-13) is phase modulated “flips” that create discrete step or step-like transitions in the waveform.

In some embodiments, the controller 248 includes a machine-learning module (not shown). In such an embodiment, the machine-learning module may be cloud-based or onboard the instrument assembly 211. The machine-learning module may be used for clot identification, treatment planning, verification of recanalization, or automated stopping criteria.

While this disclosure has been illustrated and described in detail in the foregoing drawings and description, the same is to be considered as illustrative and not restrictive in character, it being understood that only illustrative embodiments thereof have been shown and described and that all changes and modifications that come within the spirit of this disclosure are desired to be protected. For example, in some embodiments, one or more of the transducer arrays 16A, 16B, 22, whether or not movable relative to the headband 12 as described above, can be configured to first determine the precise location(s) of the clot(s), and to then treat the located clot(s) by fractionation as described above. In such embodiments, the memory 50 will include instructions executable by the processor(s) or controller(s) 48 to cause the processor(s) or controller(s) 48 to make such determinations.

What is claimed is:

1. A histotripsy device, comprising:
a first arm and a second arm spaced apart from the first arm, the first and second arms configured to be coupled to a cranium of a human or animal to locate the cranium between the first arm and the second arm,
a first probe unit mounted to the first arm and positioned such that, with the first and second arms coupled to the cranium, the first probe unit is positioned over and in contact with one of a left and right temporal region of the cranium, the first probe unit having a first transducer array configured to emit focused radiation at an ultrasonic frequency or frequency range and a first transcranial doppler probe moveable relative to the first transducer array and configured to emit sound waves,
at least one processor, and
at least one memory having instructions stored therein executable by the at least one processor to cause the at least one processor to activate the first transcranial doppler probe to produce the sound waves to determine a location of a blood clot and, in response to determining the location of the blood clot, to activate the first transducer array to produce at least one pulse of ultrasonic radiation to treat the blood clot.

2. The histotripsy device of claim 1, further comprising a second probe unit mounted to the second arm and positioned such that, with the first and second arms coupled to the cranium, the second probe unit is positioned over and in contact with the other of the left and right temporal region of the cranium, the second probe unit having a second transducer array configured to emit focused radiation at an ultrasonic frequency or frequency range and a second transcranial doppler probe moveable relative to the second transducer array and configured to emit sound waves.

3. The histotripsy device of claim 1, wherein the first transducer array includes a central through hole, and wherein the first transcranial doppler probe is moveable through the central through hole toward and away from the second arm.

4. The histotripsy device of claim 3, wherein the first transcranial doppler probe has six degrees of freedom.

5. The histotripsy device of claim 1, further comprising a headrest located between the first arm and the second arm, and wherein the first arm and the second arm are both moveably coupled to the headrest to move toward and away from the headrest.

6. The histotripsy device of claim 5, wherein the first arm includes a first sensor, and wherein the at least one processor is configured to receive inputs from the first sensor indicative of a pressure between the first probe unit and the cranium, and wherein the at least one memory has instructions stored therein executable by the at least one processor to cause the at least one processor to move the first arm relative to the headrest based on the inputs from the first sensor.

7. The histotripsy device of claim 1, wherein the first transducer array includes a plurality of annular segments that each extend around a central axis.

8. The histotripsy device of claim 7, wherein the plurality of annular segments includes seven annular segments.

9. The histotripsy device of claim 7, wherein the at least one memory has instructions stored therein executable by the at least one processor to cause the at least one processor to sequentially activate each of the plurality of annular segments included in the first transducer array to sequentially produce at least one pulse of ultrasonic radiation from each of the plurality of annular segments.

10. The histotripsy device of claim 1, wherein the at least one memory has instructions stored therein executable by the at least one processor to cause the at least one processor to activate the first transducer array to produce the at least one pulse of ultrasonic radiation having a duration of 1 milliseconds or longer, and to activate the first transducer array to produce between 500 and 1000 pulses.

11. The histotripsy device of claim 10, wherein the first transducer array is a spherically focused, multi-segment transducer with each segment having a 7.5 centimeter focal length.

12. The histotripsy device of claim 10, wherein the at least one memory has instructions stored therein executable by the at least one processor to cause the at least one processor to activate the first transducer array to produce the at least one pulse of ultrasonic radiation for a total insonation time of between 20 and 60 seconds.

13. A histotripsy device, comprising:
a first arm configured to be coupled to a cranium of a human or animal,
a first probe unit mounted to the first arm and positioned such that, with the first arm coupled to the cranium, the first probe unit is positioned over and in contact with one of a left and right temporal region of the cranium, the first probe unit having a first transducer array configured to emit focused radiation at an ultrasonic frequency or frequency range and a first transcranial doppler probe configured to emit sound waves, the first transducer array including seven annular segments that each extend around a central axis,
at least one processor, and
at least one memory having instructions stored therein executable by the at least one processor to cause the at least one processor to activate the first transcranial doppler probe to produce the sound waves to determine a location of a blood clot and, in response to determining the location of the blood clot, to activate the first transducer array to direct at least one pulse of ultrasonic radiation into the blood clot.

14. The histotripsy device of claim 13, further comprising a second arm spaced apart from the first arm and a second probe unit mounted to the second arm and positioned such that, with the first and second arms coupled to the cranium, the second probe unit is positioned over and in contact with the other of the left and right temporal region of the cranium, the second probe unit having a second transducer array configured to emit focused radiation at an ultrasonic frequency or frequency range and a second transcranial doppler probe configured to emit sound waves.

15. The histotripsy device of claim 14, wherein the first arm includes a first sensor, and wherein the at least one processor is configured to receive inputs from the first sensor indicative of a pressure between the first probe unit and the cranium, and wherein the at least one memory has instructions stored therein executable by the at least one processor to cause the at least one processor to move the first arm relative to the second arm based on the inputs from the first sensor.

16. The histotripsy device of claim 13, wherein the first transducer array includes a central through hole at the central axis, and wherein the first transcranial doppler probe is moveable through the central through hole toward and away from the first transducer array.

17. The histotripsy device of claim 13, wherein the at least one memory has instructions stored therein executable by the at least one processor to cause the at least one processor to sequentially activate each of the annular segments included in the first transducer array to sequentially produce at least one pulse of ultrasonic radiation from each of the annular segments.

18. The histotripsy device of claim 13, wherein the at least one memory has instructions stored therein executable by the at least one processor to cause the at least one processor to modify a focal point of the first transducer array based, at least in part, on inputs from the first transcranial doppler probe related to the location of the blood clot.

19. A method comprising:
positioning a cranium of a human or animal between a first arm and a second arm of a histotripsy device,
activating a first transcranial doppler probe coupled to the first arm to detect a location of a blood clot, and
based on the location of the blood clot, and without moving the cranium, activating a first transducer array coupled to the first arm to direct at least one pulse of ultrasonic radiation toward the blood clot.

20. The method of claim 19, wherein activating the first transducer array includes sequentially activating a plurality of annular segments of the first transducer array.
